(19) United States
(12) Patent Application Publication
(10) Pub. No.: US 2024/0173339 A1
(43) Pub. Date: May 30, 2024

(54) CANNABINOID FORMULATION FOR MANAGEMENT OF DEPRESSION, ANXIETY AND PTSD, AND CANNABINOID FORMULATION AS A SLEEP AID

(71) Applicant: Zyus Life Sciences Inc., Saskatoon (CA)

(72) Inventor: Brenton Harold ZETTL, Saskatoon (CA)

(21) Appl. No.: 18/552,476

(22) PCT Filed: Nov. 10, 2021
(86) PCT No.: PCT/CA2021/051602

§ 371 Date: Sep. 26, 2023

Related U.S. Application Data
(60) Provisional application No. 63/168,868, filed on Mar. 31, 2021.
(60) Provisional application No. 63/186,955, filed on May 11, 2021.

(51) Int. Cl. A61K 31/00; A61P 25/20; A61P 25/22; A61P 25/24

(52) U.S. Cl. CPC A61K 31/658; A61P 25/20; A61P 25/22; A61P 25/24

(57) ABSTRACT

Formulations and methods for managing depression, anxiety and/or PTSD are described, for use by individuals in need thereof. The formulations comprise a plurality of cannabinoids and one or more excipient, diluent or carrier. The cannabinoids are present in the formulation in optimized amounts to address these conditions in an amount of: tetrahydrocannabinol (THC), cannabichromene (CBC), and cannabidiol (CBD) as primary cannabinoids in a weight ratio of from 0-1:4-20:10-25, respectively, such as 1:5:20, 0:20:10 or 0:10:20. Formulations and methods for aiding sleep are also described, for use by individuals in need thereof, such as individuals with insomnia. The formulation comprises a plurality of cannabinoids and one or more excipient, diluent or carrier. The formulation comprises tetrahydrocannabinol (THC), cannabichromene (CBC), and cannabidiol (CBD) as primary cannabinoids in a weight ratio of from 1:0-5:15-25, respectively, such as 1:3:20, 1:0:20 or 1:4:20.

### CROSS REFERENCE TO RELATED APPLICATIONS

This application claims the benefit of and priority to U.S. Provisional Patent Application No. 63/168,868 filed Mar. 31, 2021, and to U.S. Provisional Patent Application No. 63/186,955 filed May 11, 2021, both of which are hereby incorporated by reference.

### FIELD

The present disclosure relates generally to formulations for medicinal use, or for non-medicinal benefit to a subject. More particularly, the present disclosure relates to a cannabinoid formulation for management of depression, anxiety and PTSD, and a cannabinoid formulation for aiding in sleep.

### BACKGROUND

Depression, Anxiety and/or PTSD

Individuals suffering from depression, anxiety and/or PTSD have limited options for management of their condition.

Cannabidiol (CBD) has been widely studied for antipsychotic-like effects in animal models and is suggested for its anxiolytic and antidepressant effects in patients with generalized social anxiety disorder (Mandolini G M, et al., 2018). CBD is also found to prevent stress-induced angiogenesis in a mouse model of chronic stress (Crippa J S, et al., 2018). The underlying pharmacological mechanism of the anxiolytic/antidepressant properties of CBD are suggested to be 5HT1A receptor-mediated neurotransmission, as well as, inhibition of anandamide metabolism and activation of TRPV1 receptor channels that facilitate CB1- and CB2-mediated responses (Crippa J S, et al., 2018). Δ⁹-THC exerts partial agonistic activity on CB1 and CB2 receptors with high binding affinity with CB1 receptor leading to its psychoactive activity. Binding to CB1 results in the activation of inwardly rectifying potassium channels, which decrease presynaptic neuron firing, and inhibition of voltage-sensitive calcium channels that decrease neurotransmitter release (Morales P et al., 2017).

Cannabichromene (CBC) is a major non-psychotropic cannabinoid naturally found in the Cannabis sativa plant. The proportion of each chemical class in the cannabis plant is, however, dependent on environmental growth conditions, geographical location, genetics, and chemotype (Lewis M A, et al., 2017). CBC has moderate affinity (Ki˜100 nanomolar) only for CB2 receptors and binds to CB1 receptors only at concentrations higher than 1 micromolar (Shinjyo N et al., 2013). The major CBC activity in the brain has been suggested to be partly dependent on indirect activation of CB1 receptor by inhibition of cellular uptake of anandamide (De Petrocellis L, et al., 2011) and activation of TRPA1 (Transient Receptor potential A1) channels (Izzo and Capasso R, 2012). CBC was found to be the most potent agonist of all the phytocannabinoids at TRPA1 channels (Maione S, et al., 2011). Evidence is accumulating that CBC has also shown anti-inflammatory effects (Izzo and Capasso R, 2012) and antidepressant effect in rodent models (Deyo and Musty, 2003).

CBD has been observed to act synergistically with Δ⁹-THC and contribute to the analgesic effect of medicinal-based cannabis extract (Russo 2011). Furthermore, an anti-epileptic effect of cannabidiol enriched cannabis extracts has been seen in children with refractory epilepsy (Crippa J A, et al., 2016). A Phase I clinical trial for administration of CBD-enriched herbal extract containing 49-THC in children with refractory epileptic encephalopathy showed an adequate safety profile in children and significant reduction of seizure frequency with half of the children becoming seizure-free (Reithmeier D et al., 2018). The agonistic activity of CBC with CB1 and CB2 receptors may offer a promising approach to potentiate the effect of other cannabinoids that exert their activities via binding and activation of CB1 and CB2 receptors.

The potential of cannabinoid combinations for medicinal uses has not been fully explored. It is desirable to provide a cannabinoid formulation for use in the management of depression, anxiety, and/or PTSD.

### Sleep Aid

Individuals suffering from insomnia, whether chronic or periodic, may experience stress, fatigue and exhaustion in their daily life. Left untreated over time, a lack of sleep reduces the quality of life, and may eventually result in depression or anxiety. There are limited options for aiding and managing lack of sleep.

Cannabinoids are a class of compounds naturally found in the Cannabis sativa plant. The proportion of each chemical class in the cannabis plant may depend on environmental growth conditions, geographical location, genetics, and chemotype (Lewis M A, et al., 2017).

Cannabidiol (CBD) has been studied for antipsychotic-like effects (Mandolini G M, et al., 2018), and effects on chronic stress (Crippa J S, et al., 2018). Physiological properties of CBD are suggested to be 5HT1A receptor-mediated neurotransmission, as well as, inhibition of anandamide metabolism and activation of TRPV1 receptor channels that facilitate CB1- and CB2-mediated responses (Crippa J S, et al., 2018).

Δ⁹-THC (THC) exerts partial agonistic activity on CB1 and CB2 receptors, leading to a psychoactive effect. Binding to CB1 has been shown to decrease neurotransmitter release (Morales P et al., 2017).

Cannabichromene (CBC) is a major non-psychotropic cannabinoid naturally found in the Cannabis sativa plant.

CBC has moderate affinity (Ki˜100 nanomolar) only for CB2 receptors and binds to CB1 receptors only at concentrations higher than 1 micromolar (Shinjyo N et al., 2013). The major CBC activity in the brain has been suggested to be partly dependent on indirect activation of CB1 receptor by inhibition of cellular uptake of anandamide (De Petrocellis L, et al., 2011) and activation of TRPA1 (Transient Receptor potential A1) channels (Izzo et al., 2012). CBC was found to be a potent agonist at TRPA1 channels (Maione S, et al., 2011). CBC has also shown anti-inflammatory effects (Izzo et al., 2012) and antidepressant effect in rodent models (Deyo and Musty, 2003).

CBD has been observed to act synergistically with Δ⁹-THC and contribute to the analgesic effect of medicinal-based cannabis extract (Russo 2011). Furthermore, an anti-epileptic effect of cannabidiol enriched cannabis extracts has been seen in children with refractory epilepsy (Crippa J A, et al., 2016). A Phase I clinical trial for administration of CBD-enriched herbal extract containing Δ⁹-THC in children with refractory epileptic encephalopathy showed an adequate safety profile in children and significant reduction of seizure frequency with half of the children becoming seizure-free (Reithmeier D et al., 2018). The agonistic activity of CBC with CB1 and CB2 receptors may offer a promising approach to potentiate the effect of other cannabinoids that exert their activities via binding and activation of CB1 and CB2 receptors.

The potential of cannabinoid combinations to aid with sleep or insomnia has not been explored. It is desirable to provide a cannabinoid formulation for use in the management and improvement of sleep.

### SUMMARY

It is an object of the present disclosure to obviate or mitigate at least one disadvantage of known drugs or formulations directed to treatment of depression, anxiety, and/or post-traumatic stress disorder.

The formulation includes cannabidiol (CBD) and cannabichromene (CBC) in amounts adequate to treat depression, anxiety, and/or PTSD, and may optionally include (−)-trans-delta-9-tetrahydrocannabinol (Δ⁹-THC, or “THC” herein). Formulations and methods are described, for treating a subject with a neurological or mental health condition such as depression, anxiety, and/or post-traumatic disorder (PTSD), including sub-categories and combinations thereof. The formulation comprises CBC and CBD as primary cannabinoids, optionally with THC as an additional primary cannabinoid, together with one or more excipient, diluent, or carrier. The primary cannabinoids in the formulation may comprise, on a weight ratio basis of the total primary cannabinoids: THC:CBC:CBD of 0-1:4-20:10-25 ratio.

Further, it is an object of the present disclosure to provide a formulation that aids or improves sleep.

The formulation includes cannabidiol (CBD) and cannabichromene (CBC) in amounts adequate to aid a subject with sleep, and may optionally include (−)-trans-delta-9-tetrahydrocannabinol (Δ⁹-THC, or “THC” herein). Aiding in sleep may comprise addressing insomnia or otherwise improving sleep quantity or quality. The formulation comprises THC, CBC and CBD as primary cannabinoids, together with one or more excipient, diluent, or carrier. The primary cannabinoids in the formulation may comprise, on a weight ratio basis of the total primary cannabinoids: THC:CBC:CBD of 1:0-5:15-25, such as 1:3:20 or 1:4:20.

Other aspects and features of the present disclosure will become apparent to those ordinarily skilled in the art upon review of the following description of specific embodiments.

### BRIEF DESCRIPTION OF THE FIGURES

Embodiments of the present disclosure will now be described, by way of example only, with reference to the attached Figures.

FIG. 1 depicts body weight effects on animals in stress vs control groups in Example 4 pertaining to a formulation for management of depression, anxiety and/or PTSD.

FIG. 2 depicts the validation of the chronic restraint stress model in Open Field Test (OFT) in Panel A; Elevated Plus Maze (EPM) test in Panels B and C; Forced Swim Test (FST) in Panel D; and Tail Suspension Test (TST) in Panel E in Example 4.

FIG. 3 depicts Elevated Plus Maze (EPM) results for vehicle versus CBC, CBD and [CBC+CBD] treatments in Example 4.

FIG. 4 illustrates the results of the Forced Swim Test (FST) for vehicle versus CBC, CBD and [CBC+CBD] treatments for no stress in Panel A, and for chronic resistance stressed (CRS) mice in Panel B in Example 4.

FIG. 5 shows anti-depressant effects of CBD and CBC based on immobility assessment in the Forced Swim Test (FST) for no stress in Panel A; immobility in CRS mice in Panel B; and distance movement in CRS mice in Panel C in Example 4.

### DETAILED DESCRIPTION

The present disclosure provides cannabinoid-containing formulations for treating or managing depression, anxiety, and/or post-traumatic stress disorder (PTSD).

The present disclosure also provides cannabinoid-containing formulations for aiding sleep.

There is provided herein a formulation for use in treating a neurological or mental health condition selected from the group consisting of depression, anxiety, post-traumatic stress disorder (PTSD), and combinations thereof, in a subject in need thereof, said formulation comprising cannabichromene (CBC), cannabidiol (CBD), and optionally tetrahydrocannabinol (THC), as primary cannabinoids, and an excipient; wherein the primary cannabinoids consist of, on a weight basis: THC:CBC:CBD, 0-1:4-20:10-25. The primary cannabinoids in the formulation may be present in amounts according to any one of the following ratios of THC:CBC:CBD of about 1:5:20; about 1:10:15; about 0.5:10:15; about 0.5:20:15; about 0:10:20; about 0:10:20; or about 0:20:10. The formulation may be prepared in a dosage form selected from the group consisting of a pill, tablet, gel capsule, syrup, oil-based spray, and liquid oil form. The formulation may provide a total amount of from about 5 mg to about 500 mg; from about 5 mg to about 100 mg; or from about 5 mg to about 50 mg of primary cannabinoid per dose. For example, the formulation may provide a total amount of from about 5 mg to about 25 mg of primary cannabinoid per dose; or may provide a total amount of from about 10 mg to about 20 mg of primary cannabinoid per dose. In an exemplary embodiment, the formulation is for use in treating depression, and the primary cannabinoids consist of, on a weight basis: THC:CBC:CBD, 0-1:10-20:10-20.

There is also provided herein a method for treating a neurological or mental health condition selected from the group consisting of depression, anxiety, post-traumatic stress disorder (PTSD), and combinations thereof, in a subject in need thereof, comprising administering to said subject an effective amount of a formulation comprising cannabichromene (CBC) and cannabidiol (CBD), and optionally tetrahydrocannabinol (THC), as primary cannabinoids, and an excipient; wherein the primary cannabinoids consist of, on a weight basis: THC:CBC:CBD in a ratio of 0-1:4-20:10-25. The primary cannabinoids in the formulation may be present in amounts according to any one of the following ratios of THC:CBC:CBD of about 1:5:20; about 1:10:15; about 0.5:10:15; about 0.5:20:15; about 0:10:20; about 0:10:20; or about 0:20:10. In this method, the formulation may be is administered in a dosage form selected from the group consisting of a pill, tablet, gel capsule, syrup, oil-based spray, and liquid oil form. The method may involve provides to the subject a total amount of from about 5 mg to about 500 mg; from about 5 mg to about 100 mg; or from about 5 mg to about 50 mg of primary cannabinoid per dose. For example, the formulation provides to the subject a total amount of from about 5 mg to about 25 mg of primary cannabinoid per dose; or may provide from about 10 mg to about 20 mg of primary cannabinoid per dose. In an exemplary embodiment of this method, the mental health condition is depression, and the primary cannabinoids consist of, on a weight basis: THC:CBC:CBD, 0-1:10-20:10-20.

Further, there is provided here the use of an effective amount of a formulation comprising cannabichromene (CBC) and cannabidiol (CBD), and optionally tetrahydrocannabinol (THC), as primary cannabinoids, and an excipient; for treating, or for preparation of a medicament for treating, a neurological or mental health condition selected from the group consisting of depression, anxiety, post-traumatic stress disorder (PTSD), and combinations thereof, in a subject in need thereof, wherein the primary cannabinoids consist of, on a weight basis: THC:CBC:CBD in a ratio of 0-1:4-20:10-25. In the use described, the primary cannabinoids may be present in the formulation in amounts according to one of the following ratios of THC:CBC:CBD: about 1:5:20; about 1:10:15; about 0.5:10:15; about 0.5:20:15; about 0:10:20; about 0:10:20; or about 0:20:10. The formulation used, or the medicament so prepared, may be for administration in a dosage form selected from the group consisting of a pill, tablet, gel capsule, syrup, oil-based spray, and liquid oil form. The formulation or medicament may provide to the subject a total amount of from about 5 mg to about 500 mg; from about 5 mg to about 100 mg; or from about 5 mg to about 50 mg of primary cannabinoid per dose. For example, the formulation or medicament may provide from about 5 mg to about 25 mg of primary cannabinoid per dose; or from about 10 mg to about 20 mg of primary cannabinoid per dose. In an exemplary embodiment of this use, the mental health condition is depression, and the primary cannabinoids consist of, on a weight basis: THC:CBC:CBD, 0-1:10-20:10-20.

There is described herein a formulation for use as a sleep aid in a subject in need thereof, said formulation comprising tetrahydrocannabinol (THC), cannabidiol (CBD), and optionally cannabichromene (CBC), as primary cannabinoids, and an excipient; wherein the primary cannabinoids consist of, on a weight basis of THC:CBC:CBD, 1:0-5:15-25. The subject in need thereof may be an individual who experiences sleep disturbance due to insomnia, stress or anxiety. The primary cannabinoids may be present in the formulation in amounts according to one of the following ratios of THC:CBC:CBD: about 1:0:20; about 1:3:20; or about 1:4:20. The formulation may be prepared in a dosage form selected from the group consisting of a pill, tablet, gel capsule, syrup, oil-based spray, and liquid oil form. The formulation may provide a total amount of from about 5 mg to about 500 mg; from about 5 mg to about 100 mg; or from about 5 mg to about 50 mg of primary cannabinoid per dose. For example, the formulation may provide a total amount of from about 5 mg to about 25 mg of primary cannabinoid per dose; or from about 10 mg to about 20 mg of primary cannabinoid per dose. The formulation be for use by a subject who experiences insomnia or sleep disruption due to nightmares, and the primary cannabinoids may consist of, on a weight basis, THC:CBC:CBD, 1:0:20.

There is also described herein a method for aiding sleep in a subject in need thereof, comprising administering to said subject an effective amount of a formulation comprising tetrahydrocannabinol (THC), cannabichromene (CBC), and cannabidiol (CBD) as primary cannabinoids, and an excipient; wherein the primary cannabinoids consist of, on a weight basis, THC:CBC:CBD in a ratio of 1:0-5:15-20. The subject in need thereof may be an individual who experiences sleep disturbance due to insomnia, stress or anxiety. According to this method, the primary cannabinoids may be present in the formulation in amounts according to one of the following ratios of THC:CBC:CBD: about 1:0:20; about 1:3:20; or about 1:4:20. The formulation used in the method may be administered in a dosage form selected from the group consisting of a pill, tablet, gel capsule, syrup, oil-based spray, and liquid oil form. The formulation used in the method may provide to the subject a total amount of from about 5 mg to about 500 mg; from about 5 mg to about 100 mg; or from about 5 mg to about 50 mg of primary cannabinoid per dose. For example, from about 5 mg to about 25 mg of primary cannabinoid per dose, or from about 10 mg to about 20 mg of primary cannabinoid per dose. The method may be for a subject who experiences insomnia or sleep disruption due to nightmares, and the primary cannabinoids may consist of, on a weight basis, THC:CBC:CBD, 1:0:20.

Further, there is described herein the use of an effective amount of a formulation comprising tetrahydrocannabinol (THC), cannabidiol (CBD), and optionally cannabichromene (CBC), as primary cannabinoids, and an excipient; for aiding sleep, or for preparation of a medicament for aiding sleep, in a subject in need thereof, wherein the primary cannabinoids consist of, on a weight basis, THC:CBC:CBD in a ratio of 1:0-5:15-25. The subject in need thereof may be an individual who experiences sleep disturbance due to insomnia, stress or anxiety. The primary cannabinoids may be present in the formulation in amounts according to one of the following ratios of THC:CBC:CBD: about 1:0:20; about 1:3:20; or about 1:4:20. The formulation or medicament may be used in a dosage form selected from the group consisting of a pill, tablet, gel capsule, syrup, oil-based spray, and liquid oil form. The formulation or medicament used may provide to the subject a total amount of from about 5 mg to about 500 mg; from about 5 mg to about 100 mg; or from about 5 mg to about 50 mg of primary cannabinoid per dose; for example from about 5 mg to about 25 mg of primary cannabinoid per dose or from about 10 mg to about 20 mg of primary cannabinoid per dose. The use may be for a subject who experiences insomnia or sleep disruption due to nightmares, and the primary cannabinoids may consist of, on a weight basis, THC:CBC:CBD, 1:0:20.

Depression, Anxiety, and/or PTSD

The formulation for managing depression, anxiety, and/or PTSD contains three primary cannabinoid ingredients, although other cannabinoids may be present as well. For the three primary cannabinoid ingredients, the ratios of the three to one another has been optimized relative to one another. The following primary cannabinoids are present in the formulation: cannabichromene (CBC), and cannabidiol (CBD), and optionally (−)-trans-delta-9-tetrahydrocannabinol (Δ⁹-THC), which is herein referred to as “THC”. The amount (percent wt/wt basis) of THC:CBC:CBD present in the formulation, expressed relative to each other as a percentage of these primary cannabinoid ingredients can be from about 0%-10% THC; 20%-80% CBC, and 20%-80% CBD.

When such ranges are expressed as ratios, an exemplary ratio of about 1:5:15 (which can also be expressed as 5%:24%:71%, when each is expressed as a percentage of the primary cannabinoids). Other ratios including all three primary cannabinoids may be 1:10:15; 0.5:7:22; or 1:15:15; for example. When THC is absent, exemplary ratios may be, for example 0:5:20, 0:10:20 or 0:20:10. Numerous ratios are encompassed, and each preferably adheres to the premise that THC, if present, is in smaller amounts than either CBC or CBD. For clarity: other cannabinoids may be present in the formulation, but are not included in the ratio as expressed above. Expressed as a percentage, the ratio of 0.5:5:20 is 2% THC, 20% CBC, 78% CBD. Variability is permitted in these ratios. In an exemplary embodiment, CBC is 2-fold greater than CBD; and THC may be absent from the formulation.

Dosages within these ratios are expressed in mg amounts, may be for example 1 mg THC, 10 mg CBC, and 10 mg CBD per dose. A different amount per dose, but in similar ratios may be amount 0.5 mg THC, 20 mg CBC, and 15 mg CBD per dose. A smaller amount per dose, but in similar ratios may be 0 mg THC, 10 mg CBC, and 5 mg CBD. A wide variety of other ratios are possible. A dose may be the amount present in one or more dosage forms (tablets or capsules), if multiples of a small sized dosage form may be consumed together to be considered a single dose, depending on the subject's requirement.

CBC may be present in the formulation in amounts higher than CBD, for example with CBC:CBD at 20:10 in a formulation with 20 mg CBC and 10 mg CBD per dose.

Primary Cannabinoids. The term “primary” is meant to indicate the cannabinoids that are primarily responsible for the intended effect of management of depression, anxiety, and/or PTSD, as described herein. CBC and CBD are primary cannabinoids in this context, and when present THC is also considered a primary cannabinoid. If another cannabinoid is present in the formulation in a lower, an approximately similar, or even a higher amount, the quantity present would not mean that the cannabinoid is a “primary” component of the formulation, although such additional primary cannabinoids may be present.

Non-Primary Cannabinoids. Cannabinoids other than CBC, CBD and THC that may be present in the composition may be considered as non-primary cannabinoids (which may be referenced herein as secondary cannabinoids, or incidentally-present cannabinoids). There is no requirement that such non-primary cannabinoids be present in or absent from the composition, and the presence of such non-primary cannabinoids would not count toward a total amount of primary cannabinoid in the formulation. For example, such cannabionoids may be naturally-occurring, plant-based cannabionoids that are incidentally present in an extract containing the primary cannabinoids.

Cannabinoid Sources. The primary cannabinoids CBC, CBD and optionally THC, may be present in the formulation from natural sources, such as from one or more cannabis plants, an in particular extracts thereof. Or the cannabinoids may be obtained from one or more isolated sources, or from a synthetic source where one or more of the desired cannabinoids is synthesized. A blend of natural and synthetic cannabinoids may be used so that a natural source with a variable content (due to growing conditions or other reasons), may be standardized to pre-determined amounts using adjustment with synthetic or isolated sources.

An extract may be obtained from a plant that is specially modified or grown under conditions conducive to production of a cannabinoid ratio particularly suited to the desired primary cannabinoid ratio, without needing to dramatically alter or supplement the amount of any of the primary cannabinoids present.

If purification of cannabinoids is desired extraction methods such as an ethanolic extraction, or a CO₂ based extraction may be used.

Plants may be bred or cultured, or growth conditions can be optimized to reflect the requisite ratio of [THC]:CBC:CBD (with square brackets indicating THC as an optional component). Further, two or more plants or extracts bearing ratios differing from the intended ratio may be combined in amounts that result in the desired pre-determined ratio.

Other cannabinoids may be incidentally present in the formulation, and if present, the quantities of such additional cannabinoid ingredients would not reduce the depression, anxiety, and/or PTSD management features of the formulation.

Managing Neurological Conditions. The uses of the formulations described herein have the following advantages: (a) broadening the applications of cannabinoids in areas pertaining to health management, and medicine, (b) it offers additive or synergistic effects at the CB1 and CB2 receptor level, (c) it reduces the required dosage of Δ⁹-THC and CBD for the relevant indications, and (d) there may be pharmacological benefits over conventional therapies, especially as compared benzodiazepines and selective serotonin reuptake inhibitors (SSRIs) as are used in PTSD management (Patel et al., 2017).

The dual agonistic activity of CBC in combination with CBD, and optionally Δ⁹-THC, suggests new applications of cannabinoid compounds to meet the unmet needs in patients suffering from depression, anxiety, PTSD, or combinations thereof, including categories of these conditions.

Medical Indications. The intended use of this formulation for individuals suffering from mental health or neurologically-related conditions, in particular pertaining to depression, anxiety, and/or PTSD.

The terms “depression, anxiety and PTSD” as used herein cover a variety of conditions including and not limited to categories thereof, such as stress disorders, generalized or social anxiety disorders (GAD/SAD), depressive disorders, anxiety disorders, phobias, bipolar disorder, chronic depression, persistent depressive (dysthymic) disorder, major depressive disorder, treatment-resistant depression, PTSD, panic disorders, and obsessive compulsive disorders (OCD), which can all be treated or managed using the formulation described herein.

Subjects and Populations. The formulation may be used by humans or by pets (companion animals such as dogs or cats), as well as for working animals such as horses.

Subjects in need of a therapeutic effect in the intended indications may use the formulation prior to, during, or after the onset of a triggering event, as the medical need arises. With PTSD, the formulation may be of benefit when an individual is triggered to re-live difficult memories. With anxiety, an individual may be aware of anxiety-invoking situations, where the formulation could be used in advance of exposure to the situation. With depression, individuals prone to depressive episodes may benefit from using the formulation in a preventative manner, or during or after depressive feelings surface.

Mode and Forms of Delivery. The formulation is amenable to oral delivery, such as in a pill, tablet, gel capsules, syrup, oil-based spray or liquid oil form. The oral form may be provided in a food or as a food supplement, which may be added to a food to be more palatable or readily consumed by a subject. Topical or nasal absorption is possible. A fat-soluble carrier, or nano- or micro-particles or emulsions may be used so that the highly fat-soluble cannabinoids can be more readily absorbed. Further to edible, oral, topical, and nasal delivery dosage forms, the formulation may be prepared as an injectable, for intravenous, intramuscular, or intraocular delivery. The formulation may be delivered in a vapor, such as by vaping, in a vaporizer or puffer, or may be heated to cause volatilization and inhalation which could be considered as inhalation, vaping or “smoking”.

Dosages. The formulation can be delivered in relative amounts ranging from about 0-1:4-20:10-25 on a weight basis of THC, CBC, to CBD, respectively. Other cannabinoids may be present in the formulation. On a per dosage basis, the total amount of primary cannabinoids may range from about 0.1 mg to about 500 mg; from about 5 mg to about 500 mg; from about 5 mg to about 100 mg; from 0.1 mg-50 mg, for example 1 mg-25 mg, or 5 mg-20 mg of primary cannabinoid per dose. If delivered in a liquid such as an oil, amounts of primary cannabinoids present on a weight/volume basis may be expressed on a mg/mL basis, such as from 0.1 mg/mL-50 mg/ml per dose, for example 1 mg/mL-25 mg/mL, or 5 mg/mL-20 mg/ml per dose. Dosages may be used as needed depending on the condition to be addressed, whether PTSD, depression and/or anxiety is experienced. An individual may have from 1 to 6 doses per day, with a frequency ranging from once per day or less (as needed) to more frequently, such as once every 4 hours.

An exemplary formulation may be a solid dosage form such as a pill, tablet, or granule-containing capsule. Alternatively, the formulation may be liquid-based, and may contain isolated or synthetic primary cannabinoids, or may be an oil-based extract of cannabis with 0-1 mg/mL Δ⁹-THC, 4-20 mg/mL CBC and 10-25 mg/ml of CBD in liquid forms such as oil, and oil-based spray, or a liquid-containing gel capsule (soft-gel capsule). If liquid-containing or gel-containing capsules are used, these may be limited in volume, for example an approximate volume of 200 μL. The milligram quantity stated above as a dosage range may be included in each such capsule, or the capsules may be formulated so as to be less concentrated in units of mg/mL. When less concentrated capsules are used, then the appropriate dosage is delivered by increasing the number of capsules consumed per dose.

Excipients and Formulation Ingredients. The formulation may incorporate any acceptable excipients known in formulating drugs or cannabinoids. Such ingredients may include starch, cellulose, alginates, colloidal silicon, lubricants such as stearates, salts, aqueous and non-aqueous (fat soluble) ingredients. The usual formulation considerations would be brought to bear, as one of skill in the art would understand.

### Sleep Aid

The formulation for aiding sleep contains three primary cannabinoid ingredients, although other cannabinoids may be present as well. For the three primary cannabinoid ingredients, the ratios of the three to one another has been optimized relative to one another. The following primary cannabinoids are present in the formulation: (−)-trans-delta-9-tetrahydrocannabinol (THC), cannabichromene (CBC), and cannabidiol (CBD). The amount (percent wt/wt basis) of THC:CBC:CBD present in the formulation, expressed relative to each other as a percentage of these primary cannabinoid ingredients can be from about 2%-5% THC; 8%-20% CBC, and 75%-90% CBD.

When such ranges are expressed as ratios, an exemplary ratio of about 1:3:20 (which can also be expressed as 4%:12%:84%, when each is expressed as a percentage of the primary cannabinoids). Other ratios including all three primary cannabinoids may be 1:4:20, THC:CBC:CBD for example. Numerous ratios are encompassed, and each preferably adheres to the premise that THC, if present, is in smaller amounts than either CBC or CBD. For clarity: other cannabinoids may be present in the formulation, but are not included in the ratio as expressed above. Expressed as a percentage, the ratio of 0.5:5:20 is 2% THC, 20% CBC, 78% CBD. Variability is permitted in these ratios. In an exemplary embodiment, CBC is 2-fold greater than CBD.

Dosages within these ratios are expressed in mg amounts, may be for example 1 mg THC, 4 mg CBC, and 20 mg CBD per dose. A different amount per dose, but in similar ratios may be amount 0.5 mg THC, 2 mg CBC, and 10 mg CBD per dose. A wide variety of other ratios are possible. A dose may be the amount present in one or more dosage forms (tablets or capsules), if multiples of a small sized dosage form may be consumed together to be considered a single dose, depending on the subject's requirement.

Primary Cannabinoids. The term “primary” is meant to indicate the cannabinoids that are primarily responsible for the intended effect of aiding and promoting sleep, as described herein. THC, CBC and CBD are primary cannabinoids in this context. If another cannabinoid is present in the formulation in a lower, an approximately similar, or even a higher amount, the quantity present would not mean that the cannabinoid is a “primary” component of the formulation, although such additional primary cannabinoids may be present.

Non-Primary Cannabinoids. Cannabinoids other than CBC, CBD and THC that may be present in the composition may be considered as non-primary cannabinoids (which may be referenced herein as secondary cannabinoids, or incidentally-present cannabinoids). There is no requirement that such non-primary cannabinoids be present in or absent from the composition, and the presence of such non-primary cannabinoids would not count toward a total amount of primary cannabinoid in the formulation. For example, such cannabionoids may be naturally-occurring, plant-based cannabionoids that are incidentally present in an extract containing the primary cannabinoids.

Cannabinoid Sources. The primary cannabinoids THC, CBC, and CBD may be present in the formulation from natural sources, such as from one or more cannabis plants, an in particular extracts thereof. Or the cannabinoids may be obtained from one or more isolated sources, or from a synthetic source where one or more of the desired cannabinoids is synthesized. A blend of natural and synthetic cannabinoids may be used so that a natural source with a variable content (due to growing conditions or other reasons), may be standardized to pre-determined amounts using adjustment with synthetic or isolated sources.

An extract may be obtained from a plant that is specially modified or grown under conditions conducive to production of a cannabinoid ratio particularly suited to the desired primary cannabinoid ratio, without needing to dramatically alter or supplement the amount of any of the primary cannabinoids present.

If purification of cannabinoids is desired extraction methods such as an ethanolic extraction, or a CO₂ based extraction may be used.

Plants may be bred or cultured, or growth conditions can be optimized to reflect the requisite ratio of THC:CBC:CBD. Further, two or more plants or extracts bearing ratios differing from the intended ratio may be combined in amounts that result in the desired pre-determined ratio.

Other cannabinoids may be incidentally present in the formulation, and if present, the quantities of such additional cannabinoid ingredients would not reduce the sleep management features of the formulation.

Medical Indications. The intended use of this formulation for individuals suffering from poor sleep or insomnia, which may be due to underlying factors such as stress, depression, or anxiety.

Subjects and Populations. The formulation may be used by humans or by pets (companion animals such as dogs or cats), as well as for working animals such as horses.

Mode and Forms of Delivery. The formulation is amenable to oral delivery, such as in a pill, tablet, gel capsules, syrup, oil-based spray or liquid oil form. The oral form may be provided in a food or as a food supplement, which may be added to a food to be more palatable or readily consumed by a subject. Topical or nasal absorption is possible. A fat-soluble carrier, or nano- or micro-particles or emulsions may be used so that the highly fat-soluble cannabinoids can be more readily absorbed. Further to edible, oral, topical, and nasal delivery dosage forms, the formulation may be prepared as an injectable, for intravenous, intramuscular, or intraocular delivery. The formulation may be delivered in a vapor, such as by vaping, in a vaporizer or puffer, or may be heated to cause volatilization and inhalation which could be considered as inhalation, vaping or “smoking”.

Dosages. The formulation can be delivered in relative amounts ranging from about 1:3-5:15-25 on a weight basis of THC, CBC, to CBD, respectively, for effective assistance in aiding sleep. Other cannabinoids may be present in the formulation. On a per dosage basis, the total amount of primary cannabinoids may range from about 0.1 mg to about 500 mg; from about 5 mg to about 500 mg; from about 5 mg to about 100 mg; or from 0.1 mg-50 mg, for example 1 mg-25 mg, or 5 mg-20 mg of primary cannabinoid per dose. If delivered in a liquid such as an oil, amounts of primary cannabinoids present on a weight/volume basis may be expressed on a mg/mL basis, such as from 0.1 mg/mL-50 mg/mL per dose, for example 1 mg/mL-25 mg/mL, or 5 mg/mL-20 mg/mL per dose. Dosages may be used as needed depending on the individuals need on a nightly basis. A dose can be taken from 1 to 2 hours prior to attempting sleep, or can be taken after an individual has unsuccessfully attempted sleep for one or more hours, or after an individual awoken and cannot return to sleep, such as in the middle of the night.

An exemplary formulation may be a solid dosage form such as a pill, tablet, or granule-containing capsule. Alternatively, the formulation may be liquid-based, and may contain isolated or synthetic primary cannabinoids, or may be an oil-based extract of cannabis with 1 mg/mL Δ⁹-THC, 3-5 mg/mL CBC and 15-25 mg/mL of CBD in liquid forms such as oil, and oil-based spray, or a liquid-containing gel capsule (soft-gel capsule). If liquid-containing or gel-containing capsules are used, these may be limited in volume, for example an approximate volume of 200 μL. The milligram quantity stated above as a dosage range may be included in each such capsule, or the capsules may be formulated so as to be less concentrated in units of mg/mL. When less concentrated capsules are used, then the appropriate dosage is delivered by increasing the number of capsules consumed per dose.

Excipients and Formulation Ingredients. The formulation may incorporate any acceptable excipients known in formulating drugs or cannabinoids. Such ingredients may include starch, cellulose, alginates, colloidal silicon, lubricants such as stearates, salts, aqueous and non-aqueous (fat soluble) ingredients. The usual formulation considerations would be brought to bear, as one of skill in the art would understand.

### EXAMPLES

Examples 1 to 4 pertain to formulations for management of depression, anxiety and/or PTSD, while Examples 5 to 8 pertain to formulations for use as a sleep aid.

### Example 1

Depression, Anxiety and/or PTSD—Formulation for Use in PTSD Management

The effects that accompany PTSD vary from individual to individual. Onset and persistence cannot always be predicted. An individual may consume orally, on an as-needed basis, a dose of the following oil-based cannabinoid formulation when symptoms happen.

The formulation comprises 1 mg/mL THC, 5 mg/mL CBC and 20 mg/mL CBD, in an oil-based liquid. When symptoms appear, the individual may take 1 mL orally. Initially, the individual may begin by consuming 1 mL of the formulation at a frequency of twice per day. The dose may be titrated to a higher amount over time as the individual becomes accustomed to the formulation, until a dose of 1 to 2 mL, taken from 4 to 6 times per day is reached. The dose may be used on an as-needed basis, for example, as unexpected episodes or symptoms occur, or may be used prophylactically when an individual expects onset of an episode due to predictable circumstances.

### Example 2

Depression, Anxiety and/or PTSD—Managing Anxiety

The onset of symptoms or effects of anxiety cannot always be anticipated in advance. However, for an individual who is aware of an upcoming stressor or triggering event that has caused symptoms of anxiety in the past, the individual may consume orally, on an as-needed basis, a dose of the following pill-based cannabinoid formulation in advance of the event.

The formulation comprises 0.5 mg THC, 10 mg CBC and 15 mg CBD, in an soft-gel capsule form. When symptoms appear, the individual may take 1 gel capsule orally. Optionally, a high-fat food may be simultaneously consumed to assist in efficiency of intestinal absorption.

### Example 3

Depression, Anxiety and/or PTSD—Formulation for Use in Management of Depression

Episodes of depression vary from individual to individual. Onset and persistence cannot always be predicted. For some, the symptoms may come and go quickly while others experience a persistent and long-term depressive episode. An individual may consume orally, on an as-needed basis, a dose of the following encapsulated oil-based cannabinoid formulation when experiencing depression.

The formulation is present in soft-gel capsules having an approximate volume of 200 μL per capsule. Each capsule comprises 5 mg CBC and 20 mg CBD (no THC). The soft-gel capsule encapsulates an oil-based liquid with a gelatin-based shell that may incorporate other commonly known gel capsule ingredients, such as glycerin or sorbitol, so as to permit ease of swallowing. When depression is occurring, the individual may take 1 capsule orally.

Initially, the individual may begin by consuming 1 capsule at a frequency of twice per day. The dose may be titrated to a higher amount over time as the individual becomes accustomed to the formulation, until a dose of 1 to 2 capsules, taken from 4 to 6 times per day is reached.

### Example 4

Depression, Anxiety and/or PTSD—Formulation for Management of Depression, Anxiety and/or PTSD

### Overview

A formulation is described for use by individuals experiencing depression, anxiety, or post-traumatic stress disorder. The formulation substantially contains cannabidiol (CBD) and Cannabichromene (CBC) in optimized amounts to manage anxiety, depression and/or PTSD. One or more minor cannabinoids and one or more excipient, diluent or carrier are included in the formulation. The types of depression and anxiety managed with the formulation include but are not limited to stress disorders, generalized or social anxiety disorders (GAD/SAD), obsessive-compulsive disorder (OCD), panic disorder, phobia, post-traumatic stress disorder, bipolar disorder, chronic depression, persistent depressive (dysthymic) disorder, major depressive disorder, and treatment-resistant depression. Methods of use of the formulation, doses and dosage forms are described.

### SUMMARY

Purpose. The objective of this study was to evaluate the effect of high CBD and CBC with minimal or no THC on depression and depressive-like behavior.

Methods. The study was conducted as follows:

D−1: Baseline test for behavioral studies.

D0-D14: Development of Depression Model by Chronic Restraint Stimulation (CRS) 1 hour per day.

D15: Treatment of Control and CRS mice (i.p injection of Cannabinoids or Vehicle).

D15: Open field test (OFT).

D16: Elevated plus maze test (EPM) (Replicate 1).

D17: Elevated plus maze test (EPM) (Replicate 2).

D18: Tail suspension test (TST).

D19: Forced swim test (FST).

D20: Euthanization of mice.

Results and Conclusions. Significant and highly significant reversion of

depressive-like behavior was observed on mice treated with CBD and CBC and improvement of normal functional performance were produced by cannabinoids.

Mixture of CBD and CBC displayed higher efficacy on depressive-like behavior, than either of the cannabinoids as an individual treatment.

In summary, mixture of cannabidiol and cannabichromene with minimal or no Δ⁹-tetrahydrocannabinol was proven to have highly potent anxiolytic and anti-depressive effect. According to the forced-swim test results, it is highly likely that cannabidiol and cannabichromene with minimal amount of Δ⁹-tetrahydrocannabinol ameliorate depressive behavior.

### Purpose of the Study

Medical cannabis provides health benefits to manage pain, anxiety, and depression (Kosiba J, et al., 2019). Studies showed that chronic stress or major depression, leads to a suppressed endocannabinoid system (eCBS) consisting of cannabinoid receptors (CB1 and CB2), their endogenous ligands, and enzymes responsible for synthesis and degradation of ligands (Meyer H C, et al., 2018). As such, exogenously administered cannabinoids became the attractive therapeutic entity to regulate eCBS for mood stabilization. Animal studies indicated that other receptor pathways such as 5HT1A receptors are involved in the antidepressant actions exerted by cannabinoids (Linge R, et al., 2016; Sales A J, et al., 2018). The typical cannabinoids produce locomotor suppression, catalepsy and hypothermia on certain doses. These effects should be taken into account when analyzing behavioral data to evaluate the antidepressant efficacy and these doses should be avoid when designing the experiments.

Cannabichromene (CBC), one of the most abundant naturally occurring non-psychoactive phytocannabinoid, exerts anti-inflammatory (DeLong G T, et al., 2010) and antidepressant-like activity in rodents (El-Alfy A T, et al., 2010) besides its typical actions such as analgesic, etc. Pharmacologically, CBC is an inhibitor of endocannabinoid reuptake and a weak inhibitor for MAGL (Izzo A A, et al., 2012), to inhibit endocannabinoid inactivation. CBC is a selective CB2 receptor agonist that may contribute to its modulatory functions on inflammation (Udoh M, et al., 2019). Dose-response curve showed CBC at 15 (Izzo A A, et al., 2012), 10, 30, 100 mg/kg (DeLong G T, et al., 2010) to be effective for anti-inflammatory; and CBC at 20 mg/kg on FST, and 40 or 80 mg/kg on TST exhibited the significant antidepressant effect (El-Alfy A T, et al., 2010). However, CBC at 80 (El-Alfy A T, et al., 2010) or 100 mg/kg (DeLong G T, et al., 2010) caused significant decreases in locomotor activity.

Cannabidiol (CBD), another abundant non-psychoactive phytocannabinoid, is confirmed to possess anti-anxiety, antinociceptive, antioxidant, antipsychotic, and anti-inflammatory effects (Mechoulam et al, 2007). Pharmacologically, CBD has a weak affinity for both CB1 and CB2 receptors, blocks the enzymatic hydrolysis and uptake of the eCBS ligands, and subsequently elevates local endocannabinoid levels (Premoli M, et al., 2019). Whether CBD exhibits anti-depressant properties via CB1 receptors is controversial; studies showed both serotonergic and glutamate cortical signaling mechanism were involved (Linge R, et al., 2016; Sales A J, et al., 2018). CBD exerted anti-anxiety actions also via 5HT1A receptor pathway (Campos A C, et al., 2012; Loflin M J, et al., 2017). Dose-response curve showed that CBD at 10 mg/kg (Sales A J, et al., 2018) and at 200 mg/kg (El-Alfy A T, et al., 2010) on FST were effective for antidepressant effects but 200 mg/kg CBD induced a decreased locomotor activity. CBD did not cause a significant change on TST at any dose. CBD at 5-20 mg/kg showed anti-inflammatory and immunomodulatory (rats) effects (Costa B, et al., 2007; Napimoga M H, et al., 2009) via TRPV1 receptor pathway. CBD at low dose (10 mg/kg) was effective for anti-anxiety but failed at high dose (100 mg/kg) (Campos A C, et al., 2012).

The objective of this study was to evaluate the effect of high CBD and CBC with minimal or no THC on depression and depressive-like behavior, anxiety and post-traumatic stress disorder (PTSD).

It was hypothesized that equipotent CBC and CBD given in combination may exert additive or synergistic antidepressant, anti-anxiety, and anti-inflammatory effects via modulating either cannabinoid receptor, or 5HT1A receptor, or TRPV1 receptor mechanism.

To date, the BALB/c strain is one of the most reliably responsive strains in baseline behavioral despair tests (FST and TST). Thus, this strain was used in this study. BALB/c mice show sensitivity to a wide range of clinically used antidepressants, which can be used as references to antidepressant effects from cannabinoids, although experiments are not designed in this way in this study. The stress-sensitivity of BALB/c has also been noted in anxiety-related test which is suitable to examine the anti-anxiety effects from cannabinoids in this study (Jacobson L H, 2007; Belzung C, 2001).

### Methods

All animal experiments were performed in accordance with the Guidelines of the Canadian Council on Animal Care and the Regulations of the University Animal Care Committee.

Mouse Species. Adult male BALB/c mice were purchased from Charles River Laboratories.

Distress Model. Adult male BALB/c mice weighing 20-30 g were used for these experiments. Total 160 animals=10 animals/group×8 treatment groups×2 replicates. The mice groups were as following:
1. Stress Group: Chronic Restraint Stimulation (CRS): N=80 mice
2. Control Group: No distress with CRS: N=80 mice

The group size was planned to be of n=10. After the exclusions, the final group sizes were n=6 to 10.

Cannabinoid Formulation. In accordance with the formulation described herein, formulations in this example were administered in amounts of from 0-1 mg/kg THC and 10-20 mg/kg CBC and 10-20 mg/kg CBD based on animal body weight (which may also be expressed as THC:CBC:CBD at a weight ratio of 0-0.1:1-2: 1-2).

### Treatments

Phase 1. The CRS or No CRS animals the following test article mixtures:

Group 1—Treatment with vehicle (0.9% saline)

Group 2—Treatment with CBD (10 mg/kg)

Group 3—Treatment with CBC (20 mg/kg)

Group 4—Co-Treatment CBD (10 mg/kg)+CBC (20 mg/kg)

Phase 2. Mice received the following test article mixtures:

Group 5: treatment with vehicle (0.9% saline)

Group 6: treatment with CBD (20 mg/kg)

Group 7: treatment with CBC (10 mg/kg)

Group 8: Co-treatment with CBD (20 mg/kg)+CBC (10 mg/kg) (No THC)

Group 9: Co-treatment with CBD (20 mg/kg)+CBC (10 mg/kg)+THC (1 mg/kg). Note that no data is reported herein for Phase 2 animals in Groups 5 to 9.

All mice followed the same study design until completion of Day 19 behavioral tests. The mice were then euthanized at Day 20 for tissue collection and further biochemical analyses of plasma level of cannabinoids and brain tissues.

### Behavioral Tests

Forced Swim Test (FST)
Elevated Plus Maze (EPM)
Open-Field Test (OFT)
Tail Suspension Test (TST)

Body Weight Monitoring. The mice were weighed upon Day 0 (prior stress), baseline behavioral assays and daily thereafter.

Statistical Analysis. All values are presented as group mean±standard error of the mean. All statistical analyses were conducted with a significance level of α=0.05, using GraphPad Prism statistical program (Version 8, GraphPad Software, Inc., San Diego, CA).

Levels of significance are reported, based on GraphPad definitions, as follows: ****p<0.0001, extremely/highly significant; ***p<0.001, extremely/highly significant; **p<0.01, very significant; *p<0.05, significant; and *p<0.1, an established trend towards significance.

An additional symbol (⋅) has been included in the significance scale, with the notion of (p<0.1), referring to 90-95% chance of the indicated effect being genuine and true effect—and, correspondently, 5-10% chance of the effect being false. The used statistical software refers to this significance level as “established trend towards significance”.

Unpaired t-test (Mann-Whitney U) test was considered for the comparison between two groups.

Nevertheless, as these comparisons are considered as “planned comparisons”, and there is ‘found variance’ combined with the small group size, a more conservative method with adjusting the p-values is recommended to be used. Therefore, Dunnett's multiple comparisons test was used as the post hoc test.

Otherwise, the guidelines given in the study protocol section of “Statistical Analysis” were followed.

### Results

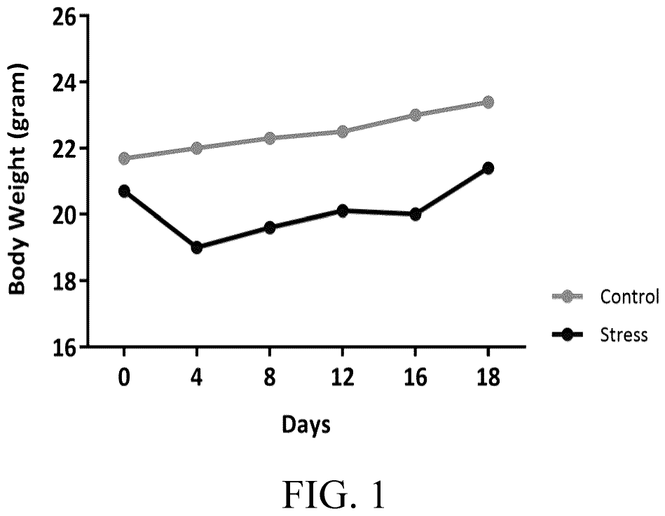

Significant Reduction of Body Weight Observed in Chronic Restraint Stress Animal Model. Body weight development of the treatment groups during the study is presented in FIG. 1. Body weights (BW) were measured upon baseline testing (BL). Thereafter, the mice were weighted daily, until the endpoint samplings on D18.

FIG. 1 shows body weight effects of animals in stress vs control groups. Data is presented as Mean+SEM (Group sizes: Control (upper), n=10; Stress (Chronic Restrained Stress (lower) or CRS, n=10).

There was a significant difference in mean BW between CRS vs. Control no distress mice. This finding confirmed the successful stress model induction. Further assessments were conducted to re-confirm successful development of stress model in mice.

Model Induction Efficacy and Result Values. Stress model induction was validated with OFT, EPM, FST and TST assessments. Overall, the data exhibited successful Stress Model Induction by Chronic restraint Stress (CRS) in BALB/c mice as confirmed by multiple measures of anxiety and depression.

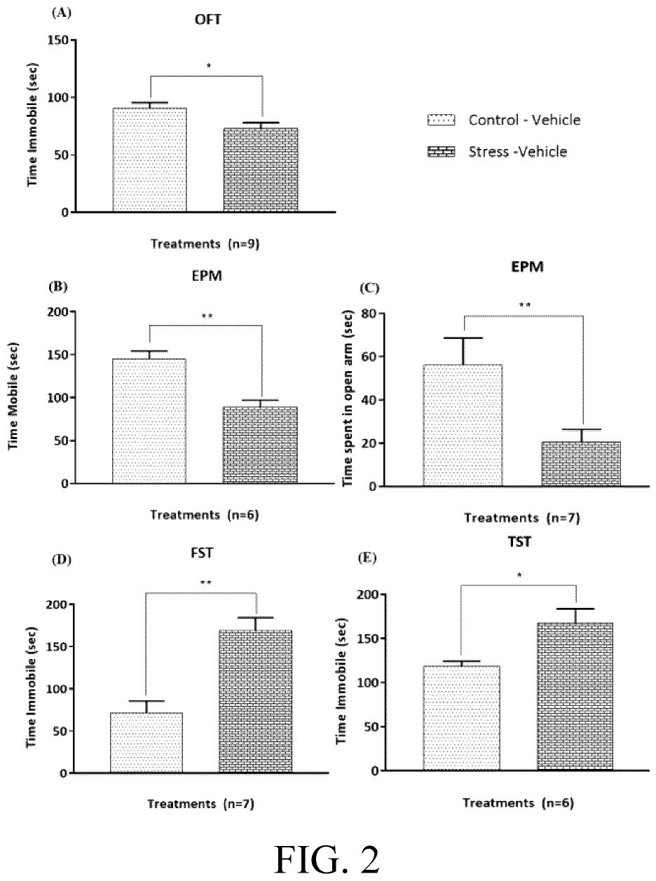

FIG. 2 shows CRS model validation. Data is presented as Mean+SEM; Group sizes are indicated as ‘n’ for Control (no stress vs. Stress group, Vehicle treatment. (Panel A) Open Field Test (OFT): Time immobile (sec) the measure of mobility, **p=0.0388; (Panels B—C) Elevated Plus Maze (EPM): Time mobile (seconds), **p=0.0043; Time spent in open arm, as a measure of anxiety-like behavior, **p=0.0042. (Panel D) Forced Swim Test (FST): Time immobile (seconds), **p=0.0012); (Panel E) Tail Suspension Test (TST) (seconds), *p=0.0260.

Measures of anxiety were evaluated by EPM and OFT test. CRS mice showed a marked reduction of mobility measured by Time Immobile (in seconds) in Open Field Test (FIG. 2, Panel A). Furthermore, Elevated Plus Maze Test (EMP) showed 40-45% reduction of mobility and 50-75% reduction in time spent in open arm, as measures of Anxiety-like behavior, in CRS group (FIG. 2, Panels B and C).

Moreover, behavioral despair tests, FST and TST, showed up to 60% and 30% increase in immobility of CRS mice, respectively (FIG. 2, Panels D and E).

These data illustrate successful Stress Model Induction by Chronic restraint Stress (CRS) in BALB/c mice as confirmed by multiple measures of anxiety and depression.

Cannabidiol and Cannabichromene Exhibit Highly Anxiolytic Effects in Mouse Model of Stress. Mice treated with cannabidiol (10 mg/kg), cannabichromene (20 mg/kg) or co-treated with cannabidiol and cannabichromene (10 and 20 mg/kg, respectively) showed a significantly higher mobility in Elevated Plus Maze (EPM). However, no significant additive effect was observed of co-administration of CBD (10 mg/kg) and CBC (20 mg/kg) compared to CBD (10 mg/kg) or CBC (20 mg/kg) as individual treatments in elevated plus maze test.

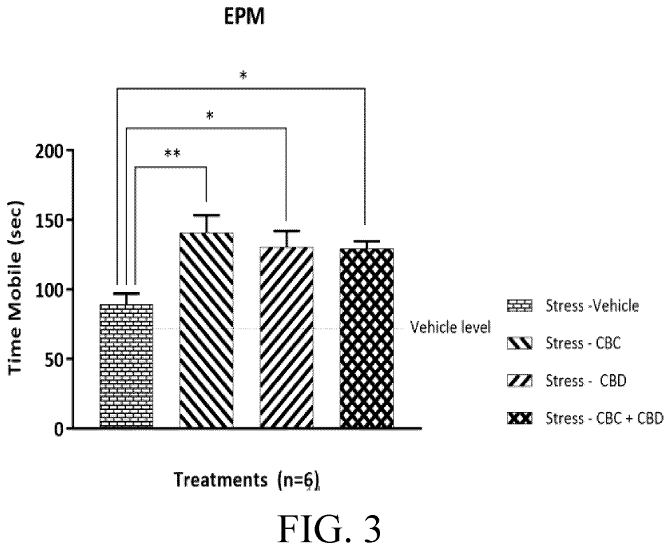

FIG. 3 shows the EPM results for vehicle versus CBC, CBD and [CBC+CBD] treatments in CRS mice.

These results demonstrated a marked anxiolytic effect of CBD and CBC. Further behavioral assays were performed to test the additive effect of these two cannabinoids as co-treatment.

### Additive Anti-Depressant Effect of Cannabidiol and Cannabichromene.

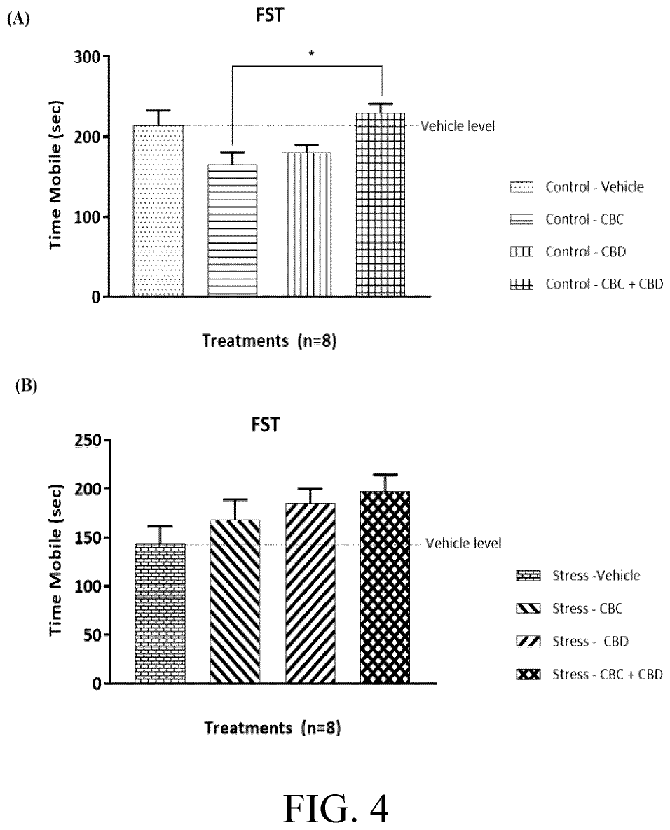

FIG. 4 the additive anti-depressant effect of CBD and CBC. Mobility assessment by Forced Swim Test (FST). Data is presented as Mean+SEM; n=number of animals per group. (Panel A) FST in Control (No Stress) mice: A significant additive anti-depressant effect of CBD (10 mg/kg)+CBC (20 mg/kg) treatment vs. individual CBC treatment of control mice: *p=0.0166; (Panel B) FST in CRS mice: A clear trend towards additive anti-depressant effect of CBD (10 mg/kg)+CBC (20 mg/kg) was detected by greater mobility time compared to Vehicle or individual CBD or CBC treatments.

Remarkably, Forced Swim Test (FST) as a widely used behavioral despair assessment, demonstrated a clear trend towards additive anti-depressive effect of CBD (10 mg/kg) and CBC (20 mg/kg) when co-administered in control or CRS mice (FIG. 4, Panel A and Panel B). In Control (No Distress) mice, CBD+CBC (10 and 20 mg/kg) co-administration displayed significantly greater mobility time compared to CBC (20 mg/kg) as an individual treatment (Panel A). In CRS mice, a clear trend was evident, as indicated by an increase mobility time in CRS mice co-treated with CBD+CBC (10 and 20 mg/kg) compared to each CBC (20 mg/kg) or CBD (10 mg/kg) administration (Panel B).

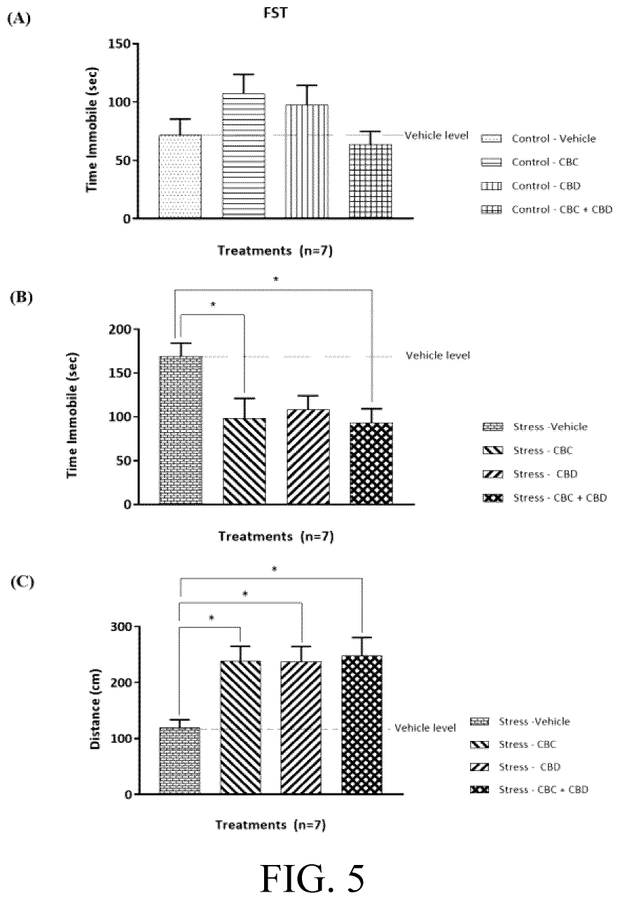

FIG. 5 shows anti-depressant effects of CBD and CBC: Immobility assessment in Forced Swim Test (FST). Data is presented as Mean+SEM; n=number of animals per group. (Panel A) Immobility in Control (No Stress) mice: A clear trend towards additive anti-depressant effect of CBD (10 mg/kg)+CBC (20 mg/kg) was detected by reduction of immobility time (seconds) as compared to individual CBD or CBC treatments. (Panel B) Immobility in CRS mice: A marked reduction of immobility was detected with CRS mice treated with CBC (20 mg/kg) (*p=0.0403) or CBD+CBC (10+20 mg/kg) compared to Vehicle (*p=0.0248). (Panel C) Distance Movement in CRS mice: CBC (20 mg/kg) vs. Vehicle: *p=0.0185; CBD (10 mg/kg) vs. Vehicle: *p=0.0194; CBC+CBD (20+10 mg/kg) vs. Vehicle: *p=0.0097.

The additive effect of CBD+CBC (10 and 20 mg/kg) was also confirmed by a clear trend towards reduction of immobility in control (no stress) mice (FIG. 5, Panel A). In Addition, a significant anti-depressive effect of CBD (10 mg/kg), CBC (20 mg/kg) or CBD+CBC (10 and 20 mg/kg) was observed, indicated by reduction of immobility time (seconds) and markedly greater movement distance (cm) in CRS mice undergoing FST (FIG. 5, Panels B and C).

### Conclusion

A BALB/c mouse model of distress was successfully developed and used in this example, with chronic restraint stress (CRS) and compared treatments with no distress BALB/c mice.

These findings confirmed an anxiolytic effect of cannabidiol and cannabichromene in a mouse model of stress, as demonstrated by elevated plus maze (EPM), a validated test for evaluation of anti-anxiety activity of drugs.

An additive anti-depressant effect of cannabidiol and cannabichromene was observed with none to minimal Δ⁹-tetrahydrocannabinol in the formulation, as indicated by Forced Swim Test (FST) in adult male BALB/c mice.

These results demonstrate a clear trend towards additive anti-depressive effect of cannabichromene and cannabidiol in the presence of no or minimal Δ⁹-THC in the Chronic Restraint Stress (CRS) mouse model. This notable reversion of depressive-like behavior in mouse model of distress was specifically important upon co-administration of cannabidiol and cannabichromene.

These findings further support the anxiolytic and anti-depressant effect of the formulation for use in in management of depression disorders, anxiety disorders, phobias, PTSD, panic disorders, and obsessive compulsive disorder (OCD).

### Example 5

### Sleep Aid—Formulation for Use in Instigating Sleep Onset

An individual may consume orally, on an as-needed basis, a dose of the following oil-based cannabinoid formulation just prior to retiring to sleep for the night.

The formulation comprises 1 mg/mL THC, 4 mg/mL CBC and 20 mg/mL CBD, in an oil-based liquid. The individual may take 1 mL orally, 1 hour before bed. Initially, the individual may begin by consuming 1 mL of the formulation on a daily basis (typically; a “nightly” basis, which can encompass night shift workers who may sleep by day). The dose may be titrated to a higher or lower amount over time as the individual becomes accustomed to the formulation, until a dose is adequate to aid in sleep. The dose may be used on an as-needed basis, for example, if an individual is experiencing recurring insomnia. The dose may be discontinued if regular and satisfactory sleep patterns are re-established.

### Example 6

### Sleep Aid—Managing Interrupted Sleep

Sleep may be interrupted in individuals in the middle of the night, and re-establishing sleep may be difficult to achieve. Sleep interruption cannot always be anticipated in advance. A dose of the formulation on an as-needed basis when sleep is interrupted can assist in aiding the individual to return to sleep.

An exemplary formulation comprising 0.5 mg THC, 1.5 mg CBC and 10; mg CBD, (a 1:3:20 ratio) may be provided in an soft-gel capsule form for consumption when sleep is interrupted, and a return to sleep is elusive after an attempt of about 10 minutes or more.

### Example 7

### Sleep Aid—Formulation for Use in Enhancing Restorative Sleep in Healthy Individuals

Not all individuals who wish to have a sleep aid are sleep deprived. Rather, all individuals may benefit from deep and restorative sleep. In anticipation of a deep restorative sleep in a healthy individual, the individual may consume orally, on an as-needed basis, a dose of the following encapsulated oil-based cannabinoid formulation when extra deep sleep is desired.

The formulation may be provided in soft-gel capsule form, having an approximate volume of 200 μL per capsule. Each capsule comprises 1 mg THC, 4 mg CBC and 20 mg CBD. The soft-gel capsule encapsulates an oil-based liquid with a gelatin-based shell that may incorporate other commonly known gel capsule ingredients, such as glycerin or sorbitol, so as to permit ease of swallowing.

### Example 8

### Sleep Aid—Enhancement of Sleep

This example shows how individuals who self-administer the formulation experienced enhanced sleep.

Testimonial of John H: A 59-year-old male with degenerative disc disease as well as neck and back injuries who has experienced chronic pain and consistent sleep disruption for over twenty years. Sleep disruption has been caused by nerve pain caused by inflammation. “I had not slept a full night in well over twenty years. Sleep is further disrupted by nightmares because of PTSD. I have been using a high CBD (THC:CBD of 1:20) oil for about 7 months. Since commencing the use of a high CBD oil (less than one ml of 1:20 oil per day) the inflammation in my neck and back has been reduced and the chronic pain has been greatly reduced. Mobility in my back and neck has increased. In the past six months I have only woken approximately five times in the middle of the night. Prior to utilizing CBD oil, I was up every night. I have not had a nightmare since beginning to take the 1:20 oil. I take 0.5 ml of 1:20 oil in the morning and 0.5 ml of 1:20 oil after dinner. Sleep ranges from 7.5 hours per night to over 12 hours. I wake in the morning feeling relaxed, refreshed and mentally alert. There is a calmness to my demeanor instead of a hyper vigilance. Traditional pharmaceutical drugs were not an option for me as I do not react well with them and am unable to function due to a level of impairment. I have had no side effects from 1:20 (THC:CBD) oil. The oil (1:20 THC:CBD) has not impaired my ability to function daily. In fact, the result is quite the opposite”. Conditions: Cervical Spondy-loarthritis, Degenerative Disc Disease Lumbar Spine.

Testimonial of Michelle G: A 47-year female with no serious condition, Ordered 1:20 (THC:CBD) oil for sleep. “I have been using the 1:20 (THC:CBD) oil product for about a week. I have been taking 0.5 ml of 1:20 formulation each night approximately ½ hour before going to bed every day. Within the first 48 hours, I experienced the following improvements in my sleep:
Muscle relaxation;
Ability to clear my mind of stress factors once I go to bed, which I was not able to do prior to using the product;
Dreams no longer focus on stressful aspects of my life;
I no longer feel like I was half awake and half asleep (or only lightly sleeping) while in bed;
Less body movement when I sleep;
Waking up refreshed, in a positive mood and with clear mind and without an alarm, whereas prior to using the product I only woke up with the assistance of an alarm and was sluggish and exhausted for at least 30 minutes after waking.
   In addition to improvements in sleep, I have also noted my neck pain is gone (which, previous to taking the product, caused me much discomfort when doing routine things such as shoulder checking when I drive)”.

In the preceding description, for purposes of explanation, numerous details are set forth in order to provide a thorough understanding of the embodiments. However, it will be apparent to one skilled in the art that these specific details are not required. References cited herein are incorporated by reference.

The above-described embodiments are intended to be examples only. Alterations, modifications and variations can be affected to the particular embodiments by those of skill in the art. The scope of the claims should not be limited by the particular embodiments set forth herein, but should be construed in a manner consistent with the specification as a whole.

### REFERENCES

The following references are incorporated by reference herein.
WO2016/044370 A1
WO2013/165251 A1
WO2012/144892 A1
WO2012/160358 A1
WO2007/083098 A1
US2016/0106705
US2016/0360721
US2018/0193304
Belzung C, G. G. (2001). Measuring normal and pathological anxiety-like behavior in mice: a review. Behav Brain Res, 1; 125(1-2): 141-9.
Campos A C, et al., (2012). Cannabidiol blocks long-lasting behavioral consequences of predator threat stress: possible involvement of 5HT1A receptors. J Psychiatr Res, 46(11):1501-10.
Costa B, et al., (2007). The non-psychoactive cannabis constituent cannabidiol is an orally effective therapeutic agent in rat chronic inflammatory and neuropathic pain. Eur J Pharmacol, 556(1-3):75-83.
Crippa J A, Crippa A C, Hallak J E, Martin-Santos R, Zuardi A W. (2016). “Δ9-THC intoxication by cannabidiol-enriched cannabis extract in two children with refractory epilepsy: full remission after switching to purified cannabidiol.” Front. Pharmacol. 7:35.
Crippa J A, Guimaraes F S, Campos A C, Zuardi A W. (2018). “Translational Investigation of the Therapeutic Potential of Cannabidiol (CBD): Toward a New Age.” Fron. Immunol. September 21; 9:02009.
DeLong G T, et al. (2010). Pharmacological evaluation of the natural constituent of Cannabis sativa, cannabichromene and its modulation by Δ(9)-tetrahydrocannabinol. Drug Alcohol Depend, 112(1-2): 126-33.
De Petrocellis L, Ligresti A, Moriello A S, Allara M, Bisogno T, Petrosino S, Stott C G, Di Marzo V. (2011). “Effects of cannabinoids and cannabinoid-enriched Cannabis extracts on TRP channels and endocannabinoid metabolic enzymes.” Br J Pharmacol 163:1479.
Deyo R, and Musty R. (2003). “A cannabichromene (CBC) extract alters behavioral despair on the mouse tail suspension test of depression.” Proceedings of 2003 Symposium on the Cannabinoids, Burlington, Vermont, International Cannabinoid Research Society, p 146.
El-Alfy A T, et al., (2010). Antidepressant-like effect of delta9-tetrahydrocannabinol and other cannabinoids isolated from Cannabis sativa L. Pharmacol Biochem Behav, 95(4):434-42.
Guimaraes F S, Chiaretti T M, Graeff F G, Zuardi A W. (1990). “Antianxiety effect of cannabidiol in the elevated plus-maze”. Psychopharmacology (1990) 100:558-9.
Izzo A A, Borrelli F, Capasso R, Di Marzo V, Mechoulam R. (2009). “Non-psychotropic plant cannabinoids: new therapeutic opportunities from an ancient herb.” Trends Pharmacol Sci 30:515.
Izzo, A A, and Aviello G, Borrelli F, Romano B, Piscitelli F, Gallo L, Capasso F, Orlando P, Di Marzo V Capasso R. (2012). “Inhibitory effect of cannabichromene, a major non-psychotropic cannabinoid extracted from Cannabis sativa, on inflammation-induced hypermotility in mice.” Br J Pharmacol 166(4): 1444-60.
Jacobson L H, C. J. (2007). Feeling strained? Influence of genetic background on depression-related behavior in mice: a review. Behav Genet. Behav Genet, 37(1): 171-213.
Kosiba J, et al, (2019). Patient-reported use of medical cannabis for pain, anxiety, and depression symptoms: Systematic review and meta-analysis. Soc Sci Med., 233:181-1.
Lewis M A, Russo E B, and Smith K M. (2017). “Pharmacological foundations of cannabis chemovars.” Planta Med. 84: 225-233.
Linge R, et al. (2016). Cannabidiol induces rapid-acting antidepressant-like effects and enhances cortical 5-H T/glutamate neurotransmission: role of 5-HT1A receptors. Neuropharmacology, 103:16-26.
Loflin M J, et al. (2017). Cannabinoids as therapeutic for PTSD. Curr Opin Psychol, 14:78-83.
Maione S, Piscitelli F, Gatta L, D. Vita, L. De Petrocellis, E. Palazzo, V. de Novellis, V. Di Marzo. (2011). “Non-psychoactive cannabinoids modulate the descending pathway of antinociception in anaesthetized rats through several mechanisms of action.” Br. J. Pharmacol. 162: 584-596.
Mandolini G M, Lazzaretti M, Pigoni A, Oldani L, et al. (2018). “Pharmacological properties of cannabidiol in the treatment of psychiatric disorders: a critical overview.” Epidemiol Psychiatr Sci. 27(4):327-335.
Mechoulam R, et al. (2007). Cannabidiol—Recent Advances. Chem Biodivers, 4(8): 1678-92.
Meyer H C, et al., (2018). The Role of the Endocannabinoid System and Genetic Variation in Adolescent Brain Development. Neuropsychopharmacology, 43(1):21-33.
Morales P, Hurst, D. P., and Reggio, P. H. (2017). “Molecular targets of the phytocannabinoids—a complex picture.” Prog. Chem. Org. Nat. Prod. 103-131.
Napimoga M H, et al., (2009). Cannabidiol decreases bone resorption by inhibiting RANK/RANKL expression and pro-inflammatory cytokines during experimental periodontitis in rats. Int Immunopharmacol, 9(2):216-22.
Patel, S., Hill, M. N., Cheer, J. F., Wotjak, C. T., and Holmes, A. (2017). “The endocannabinoid system as a target for novel anxiolytic drugs.” Neurosci. Biobehav. Rev. 76: 56-66.
Premoli M, et al. (2019). Cannabidiol: Recent advances and new insights for neuropsychiatric disorders treatment. Life Sci, 224:120-127.
Reithmeier D, Tang-Wai R, Seifert B, Lyon A W, Alcorn J, Acton B, Corley S, Prosser-Loose E, Mousseau D D, Lim H J, Tellez-Zenteno J, Huh L, Leung E, Carmant L and Huntsman R J. (2018). “The protocol for the Cannabidiol in children with refractory epileptic encephalopathy (CARE-E) study: a phase 1 dosage escalation study.” BMC Pediatrics 18:221.
Russo, E B. (2011). “Taming THC: potential cannabis synergy and phytocannabinoid-terpenoid entourage effects.” Br J Pharmacol 163:1344.
Sales A J, et al. (2018). Antidepressant-like effect induced by Cannabidiol is dependent on brain serotonin levels. Prog Neuropsychopharmacol Biol Psychiatry., 1.86:255-26.
Shinjyo N, Di Marxo V. (2013). “The effect of cannabichromene on adult neural stem/progenitor cells.” Neurochemistry International 63(5): 432-437.
Udoh M, et al., (2019). Cannabichromene is a cannabinoid CB2 receptor agonist. Br J Pharmacol. December; 176(23):4537-4547.
Wolf S A, Bick-Sander A, Fabel K, Leal-Galicia P, Tauber S, Ramirez-Rodriguez G, et al. (2010). “Cannabinoid receptor CB1 mediates baseline and activity-induced survival of new neurons in adult hippocampal neurogenesis.” Cell Commun Signal. 8:12.

1. A formulation for use in treating a neurological or mental health condition selected from the group consisting of depression, anxiety, post-traumatic stress disorder (PTSD), and combinations thereof, in a subject in need thereof, said formulation comprising cannabichromene (CBC), cannabidiol (CBD), and optionally tetrahydrocannabinol (THC), as primary cannabinoids, and an excipient;
wherein the primary cannabinoids consist of, on a weight basis: THC:CBC:CBD, 0-1:4-20:10-25.

2. The formulation for use according to claim 1, wherein the mental health condition to be treated comprises post-traumatic stress disorder (PTSD).

3. The formulation for use according to claim 1, wherein the mental health condition to be treated comprises anxiety.

4. The formulation for use according to claim 1, wherein the mental health condition to be treated comprises depression.

5. The formulation for use according to claim 1, wherein the primary cannabinoids are present in amounts according to any one of the following ratios of THC:CBC:CBD:
about 1:5:20;
about 1:10:15;
about 0.5:10:15;
about 0.5:20:15;
about 0:10:20;
about 0:10:20; or
about 0:20:10.

6. The formulation for use according to claim 1, wherein the formulation is prepared in a dosage form selected from the group consisting of a pill, tablet, gel capsule, syrup, oil-based spray, and liquid oil form.

7. The formulation for use according to claim 1, wherein the formulation provides a total amount of from about 5 mg to about 500 mg; from about 5 mg to about 100 mg; or from about 5 mg to about 50 mg of primary cannabinoid per dose.

8. The formulation for use according to claim 1, wherein the formulation provides a total amount of from about 5 mg to about 25 mg of primary cannabinoid per dose.

9. The formulation for use according to claim 8, wherein the formulation provides a total amount of from about 10 mg to about 20 mg of primary cannabinoid per dose.

10. A method for treating a neurological or mental health condition selected from the group consisting of depression, anxiety, post-traumatic stress disorder (PTSD), and combinations thereof, in a subject in need thereof, comprising administering to said subject an effective amount of a formulation comprising cannabichromene (CBC) and cannabidiol (CBD), and optionally tetrahydrocannabinol (THC), as primary cannabinoids, and an excipient;
wherein the primary cannabinoids consist of, on a weight basis: THC:CBC:CBD in a ratio of 0-1:4-20:10-25.

11. The method according to claim 10, wherein the condition to be treated comprises post-traumatic disorder (PTSD).

12. The method according to claim 10, wherein the condition to be treated comprises anxiety.

13. The method according to claim 10, wherein the condition to be treated comprises depression.

14. The method according to claim 10, wherein the primary cannabinoids are present in the formulation in amounts according to one of the following ratios of THC:CBC:CBD:
about 1:5:20;
about 1:10:15;
about 0.5:10:15;
about 0.5:20:15;
about 0:10:20;
about 0:10:20; or
about 0:20:10.

15. The method according to claim 10, wherein the formulation is administered in a dosage form selected from the group consisting of a pill, tablet, gel capsule, syrup, oil-based spray, and liquid oil form.

16. The method according to claim 10, wherein the formulation provides to the subject a total amount of from about 5 mg to about 500 mg; from about 5 mg to about 100 mg; or from about 5 mg to about 50 mg of primary cannabinoid per dose.

17. The method according to claim 10, wherein the formulation provides to the subject a total amount of from about 5 mg to about 25 mg of primary cannabinoid per dose.

18. The method according to claim 17, wherein the formulation provides to the subject a total amount of from about 10 mg to about 20 mg of primary cannabinoid per dose.

19. (canceled)

20. (canceled)

21. (canceled)

22. (canceled)

23. (canceled)

24. (canceled)

25. (canceled)

26. (canceled)

27. (canceled)

28. The formulation for use according to claim 1,
wherein the mental health condition is depression, and the primary cannabinoids consist of, on a weight basis: THC:CBC:CBD, 0-1:10-20:10-20.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. The method according to claim 10, wherein the mental health condition is depression, and the primary cannabinoids consist of, on a weight basis: THC:CBC:CBD, 0-1:10-20:10-20
